(19) United States
(12) Patent Application Publication
(10) Pub. No.: US 2016/0339123 A1
(43) Pub. Date: Nov. 24, 2016

(54) POLYBUTYLACRYLATE-BASED NANOCOMPOSITE PHOTOACOUSTIC IMAGING AGENT AND A PREPARATION METHOD THEREOF

(71) Applicant: Wenzhou Institute of Biomaterials and Engineering, Wenzhou City (CN)

(72) Inventors: Zhe Liu, Wenzhou City (CN); Yihong Li, Wenzhou City (CN); Yuanhui Song, Wenzhou City (CN); Changcan Shi, Wenzhou City (CN); Jie Wang, Wenzhou City (CN)

(21) Appl. No.: 14/716,893

(22) Filed: May 20, 2015

(51) Int. Cl. A61K 49/00

(52) U.S. Cl. CPC A61K 49/0054; A61K 49/0034; A61K 49/003

(57) ABSTRACT

A polybutylacrylate-based nanocomposite photoacoustic imaging agent includes a biomedical material such as polybutylacrylate used as a main component and methylene blue or indocyanine green used as an entrapped component with particle diameters of 525.1 nm and 272.0 nm and electric potentials of −4.52 mV and −5.98 mV respectively. The preparation method of the polybutylacrylate-based nanocomposite photoacoustic imaging agent with the features of moderate eco-friendliness, low power consumption, and free of three industrial wastes, radiation, and noise pollution may be used for industrial production.

### BACKGROUND OF THE INVENTION

(a) Technical Field of the Invention

The present invention relates to a polybutylacrylate-based nanocomposite photoacoustic imaging agent and its preparation method.

(b) Description of the Prior Art

Photoacoustic Imaging (PAI) is a biomedical imaging technology has emerged in the past decade and is an imaging method that receives a response to photoacoustic wave signals generated by a radiation medium of an excitation light (which is the photoacoustic effect) to obtain the tomography of biological tissues and 3D images and concurrently has the advantages of the high sensitivity of optical imaging and the deep penetration of acoustic imaging, so that PAI is one of the imaging modes with the broad prospect of application for the high-resolution high-contrast imaging of deep tissues. Since a variety of endogenous biochemical substances such as deoxy-hemoglobin, melanin, fat and water in living organisms may be excited by an excitation light of a specific wavelength, and these ingredients are closely related to physiological functions, therefore the photoacoustic imaging is capable of reflecting the physiological structure of a living organism and providing abundant bioinformatics including anatomy, function, metabolism, molecule and genes. The photoacoustic imaging has been applied on imaging brain tissues and monitoring tumor angiogenesis. However, the light scattering effect may increases the ratio of light intensity to photoacoustic noise with the depth of biological tissues, and shows an exponential decay, and thus giving rise to the issue of lowering the imaging resolution. Recently, the research and development of photoacoustic imaging agents gain increasingly more attention, and the acoustic and optical properties of some tissues may be changed by an exogenous imaging agent to further improve the contrast and resolution of the photoacoustic imaging, so that PAI shows a tremendous prospect of application in biological imaging and clinical and translational medicine.

At present, common photoacoustic imaging agents include nanogold particles, monolayer nanocarbon tubes and some other related nanomaterials. These materials have the advantages of small particle diameter and high stability and the disadvantages of relatively lower biocompatibility and in vivo biodegradability, greater cytotoxicity, and shorter half life which limit the scope of applicability. Since near infrared fluorescence dyes and photoacoustic probes for organic polymers have become popular research subjects, wherein methylene blue and indocyanine green have been certified as photoacoustic imaging dyes by U.S. Food and Drug Administration (USFDA). The absorption peak of methylene blue is approximately equal to 664 nm, and the absorption peak of indocyanine green approximately equal to 780 nm, and both absorption peaks falling within the near infrared region give a good foundation for their extensive application. However, these two dyes have the deficiency of relatively lower stability and shorter life cycle and require the entrapped carrier of dye molecules for clinical applications.

Polybutylacrylate is a common biomedical material used extensively in medical and pharmaceutical fields. Polybutylacrylate is often used for preparing pharmaceutical intermediates, clinical hemostats, etc. Polybutylacrylate is also a FDA certified medical adhesive and an effective composition for the treatment of cerebral arteriovenous malformation embolization and having good biocompatibility and in vivo biodegradability.

### SUMMARY OF THE INVENTION

In view of the aforementioned drawbacks, it is a first objective of the present invention to overcome the drawbacks by providing a polybutylacrylate-based nanocomposite photoacoustic imaging agent with good biocompatibility and safety and high sensitivity which may be applied more extensively in the fields biomedical imaging, targeted diagnosis and treatment, drug screening and optimization, and in vivo marker and tracking, and has potential values in individualized medicine.

A second objective of the present invention is to provide a preparation method of a polybutylacrylate-based nanocomposite photoacoustic imaging agent with the features of moderate eco-friendliness, low power consumption, and free of three industrial wastes, radiation, and noise pollution, simple and easy separation and purification process, and high yield and entrapping rate, so that the method may be used for industrial production.

To achieve the first objective, the present invention provides a polybutylacrylate-based nanocomposite photoacoustic imaging agent comprising polybutylacrylate as a main component, and methylene blue or indocyanine green as an entrapped component.

Wherein, the entrapped component is methylene blue with a particle diameter of 525.1 nm and an electric potential of −4.52 mV, and the methylene blue in the nanocomposite photoacoustic imaging agent has a concentration of 7.6 μg/mL.

Wherein, the entrapped component is indocyanine green with a particle diameter of 272.0 nm and an electric potential of −5.98 mV, and the indocyanine green in the nanocomposite photoacoustic imaging agent has a concentration of 5.8-6 μg/mL.

To achieve the second objective, the present invention provides a preparation method of a polybutylacrylate-based nanocomposite photoacoustic imaging agent comprising the following steps:

(a) Put 1.5 mL of butylacrylate, 100 mL of dextran solution, and 6.4 mg of methylene blue or 4.9 mg of indocyanine green, or these ingredients in their equivalent proportion into a reaction system.

(b) Adjust the pH value of the reaction system to 2.0˜3.0, and add dilute sodium hydroxide solution after mixing the ingredients at room temperature, and terminate the reaction.

(c) Let the reaction system sit still for 4˜7 hours, and transfer the reaction solution to a centrifuge tube for a centrifugal purification.

(d) Discard a supernant liquid, rinse by highly pure water, vortex for several times, and repeat the centrifuge step for at least two times.

(e) Obtain the nanocomposite photoacoustic imaging agent after a centrifugal concentration.

The nanocomposite photoacoustic imaging agent of the present invention has great significance on the subjects of developing medical imaging agents and systems, expanding the preparation of photoacoustic imaging agents, and providing a broad scope of applicability in the fields of photoacoustic imaging and biomedicine.

After the separation and purification of the nanocomposite photoacoustic imaging agent entrapped by methylene blue or indocyanine green in accordance with the present invention, the yield, appearance and structure of the photoacoustic imaging agent can be analyzed by a scanning electron microscope or a transmission electron microscope (as shown in FIG. 3), and the particle diameter, size distribution, and electric potential of the photoacoustic imaging agent can be obtained by a laser particle size analyzer (as shown in FIG. 4). In the photoacoustic field, photoacoustic wave signals generated by the excitation of near infrared light, and the reconstruction of the response to probe collect signals are shown in FIG. 5. Compared with a control (without being entrapped by polybutylacrylate nanoparticles), the nanocomposite photoacoustic imaging agent entrapped by methylene blue or indocyanine green enhances the contrast and resolution of the photoacoustic imaging significantly and has broad prospect of application in the biomedical imaging field.

### BRIEF DESCRIPTION OF THE DRAWINGS

FIG. 1 shows a general structure of a nanocomposite photoacoustic imaging agent of the present invention;

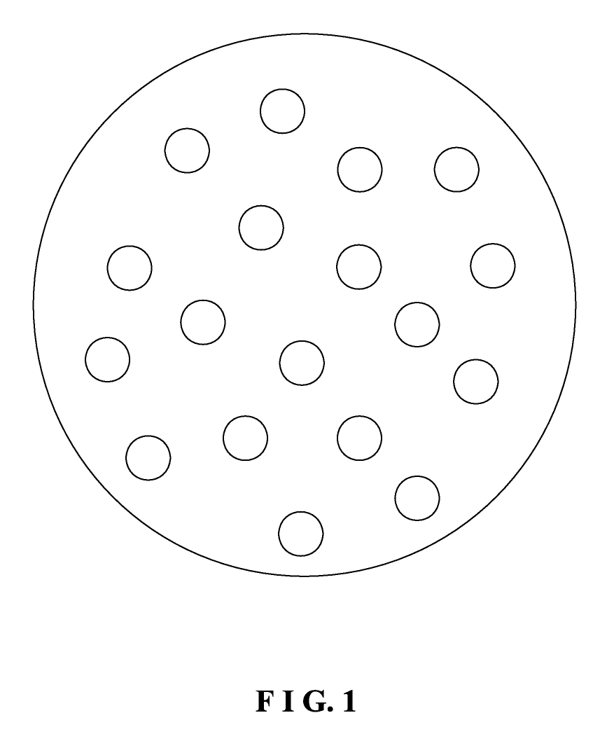

FIG. 2 is a flow chart of a preparation method of a nanocomposite photoacoustic imaging agent of the present invention;

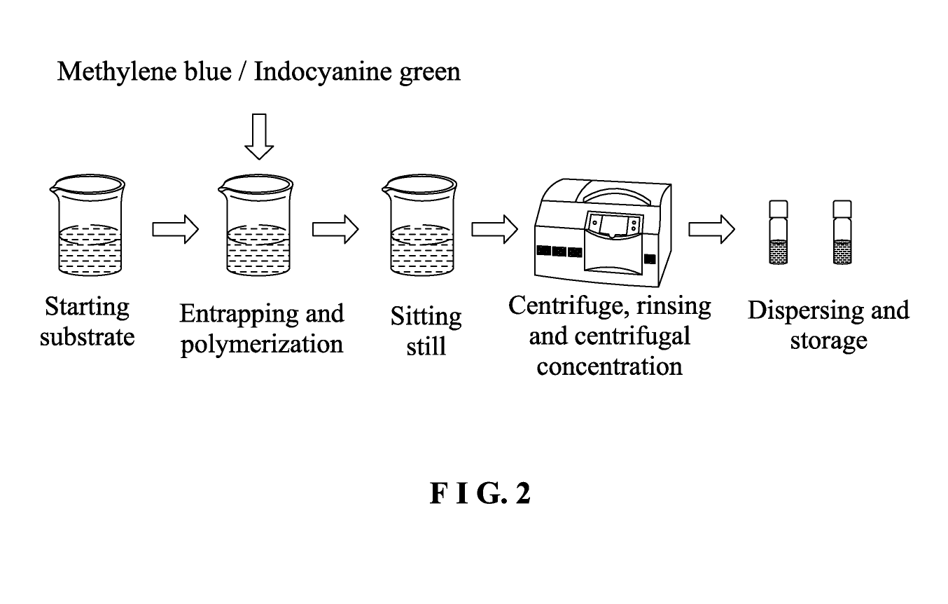

FIG. 3 shows a nanocomposite photoacoustic imaging agent (PBCA-MB) entrapped by methylene blue analyzed by a scanning electron microscope (shown in the figure on the left side) and by a transmission electron microscope (shown in the figure on the right side);

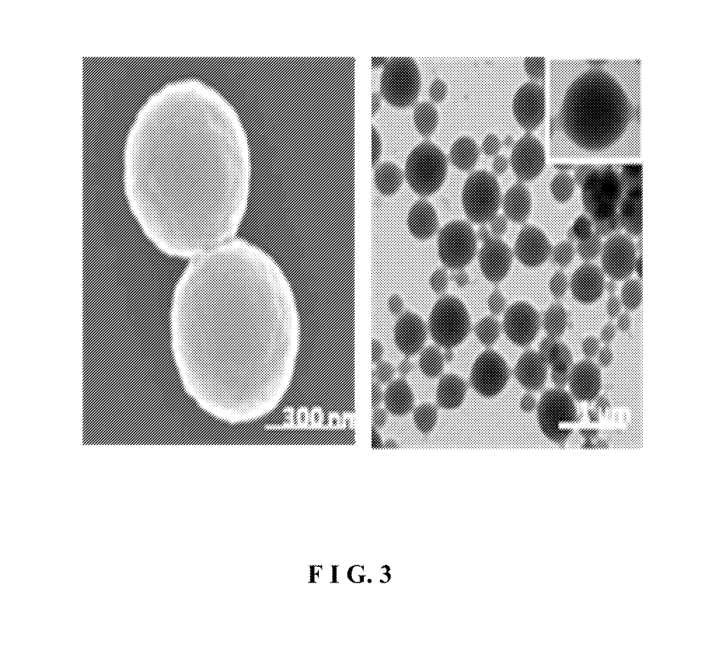

FIG. 4 shows the size distribution by intensity and the electric potential distribution of a nanocomposite photoacoustic imaging agent (PBCA-MB) entrapped by methylene blue (shown in the figures above) and a nanocomposite photoacoustic imaging agent (PBCA-ICG) entrapped by indocyanine green (shown in the figures below); and

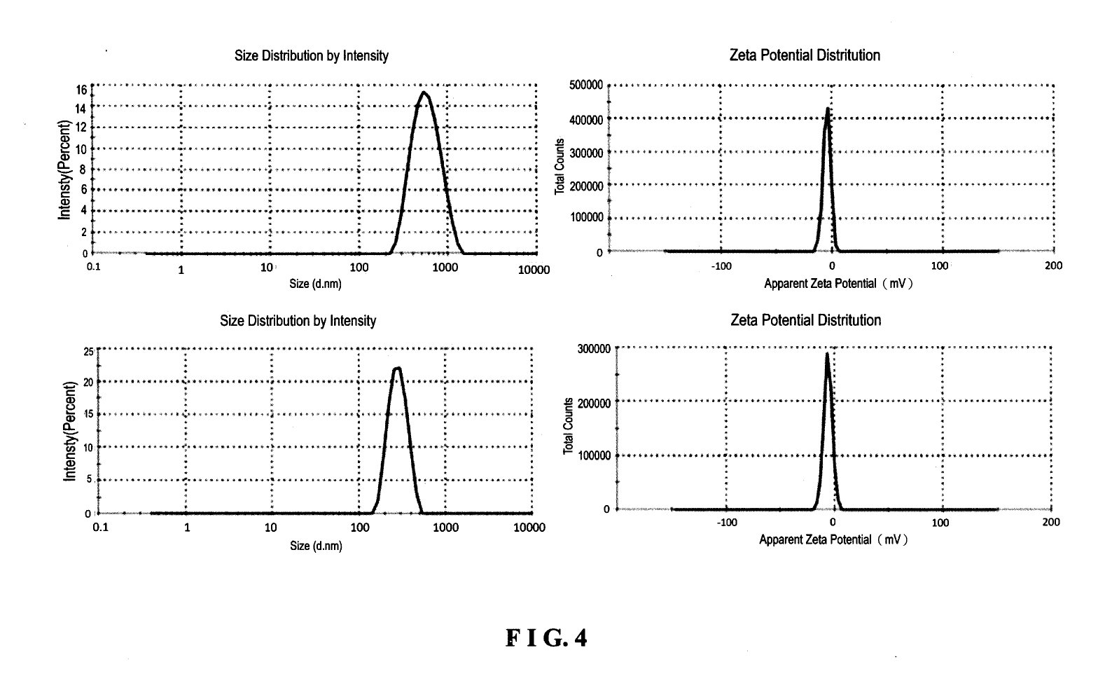

FIG. 5 shows a nanocomposite photoacoustic imaging agent (PBCA-MB) entrapped by methylene blue (shown in the figures in the middle), a nanocomposite photoacoustic imaging agent (PBCA-ICG) entrapped by indocyanine green (shown in the figures below), and a control (shown in the figures above).

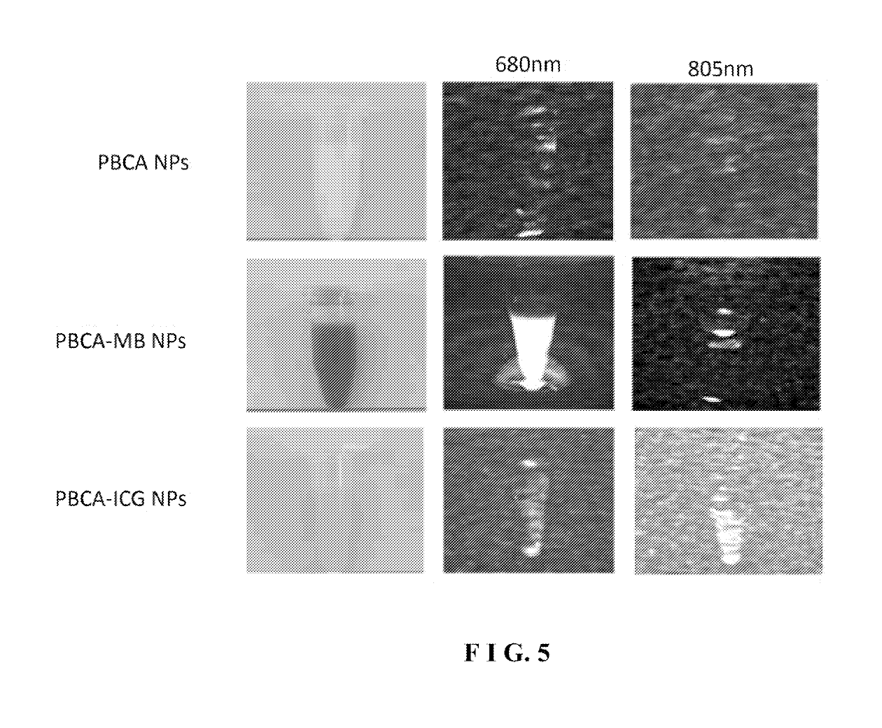

### DETAILED DESCRIPTION OF THE PREFERRED EMBODIMENTS

The present invention will become clearer in light of the following detailed description of an illustrative embodiment of this invention described in connection with the drawings. It is intended that the embodiments and drawings disclosed herein are to be considered illustrative rather than restrictive.

The materials used for preparing the photoacoustic imaging agent of the present invention are easily available in the market.

In a first preferred embodiment, butyl acrylate (1.5 mL), dextran solution (100 mL), methylene blue (6.4 mg) or these ingredients in their equivalent proportion are put into a 250 mL beaker, and the pH value of the reaction system is adjusted to 2.0˜3.0 by 1M hydrochloric acid (HCL) solution. After mixing the reaction system at room temperature, a dilute sodium hydroxide solution is added, and the reaction is terminated. The reaction system sits still for 4˜7 hours, and then all of the reaction solution are transferred to a 50 mL centrifuge tube for centrifugal purification. A supernant liquid is discarded, and the reaction solution is rinsed with highly pure water, vortexed for several times, and the centrifuge steps are repeated for at least two times. A pure nanocomposite photoacoustic imaging agent is obtained after a centrifugal concentration. The pure nanocomposite photoacoustic imaging agent may be stored in vacuum freezing or dispersed in highly pure water at room temperature. The methylene blue in the nanocomposite photoacoustic imaging agent has a concentration of 7.6 μg/mL. Measured by a laser particle size analyzer through the dynamic scattering principle, the nanocomposite photoacoustic imaging agent entrapped by methylene blue has a particle diameter of 525.1 nm, and a surface electric potential of −4.52 mV. The clear images of the microscopic surface and the fine structure of the nanocomposite photoacoustic imaging agent can be obtained and analyzed by a scanning electron microscope or a transmission electron microscope. Compared with a control of a non-entrapped nanocomposite photoacoustic imaging agent, the imaging and developing effects of the nanocomposite photoacoustic imaging agent of the present invention shows much better contrast and resolution of the photoacoustic imaging in a photoacoustic field. A stronger photoacoustic response signal is generated in a very low concentration, and thus the invention may be applied in the fields of biomarker and medical imaging.

In a second preferred embodiment, butyl acrylate (1.5 mL), dextran solution (100 mL), and indocyanine green (4.9 mg) or these ingredients in their equivalent proportion are put into a 250 mL beaker, and the pH value of the reaction system is adjusted to 2.0˜3.0 by 1M hydrochloric acid. After mixing the reaction system at room temperature, a dilute sodium hydroxide solution is added, and the reaction is terminated. The reaction system sits still for 4˜7 hours, and then all of the reaction solution are transferred to a 50 mL centrifuge tube for centrifugal purification. A supernant liquid is discarded, and the reaction solution is rinsed with highly pure water, vortexed for several times, and the centrifuge steps are repeated for at least two times. A pure nanocomposite photoacoustic imaging agent is obtained after a centrifugal concentration. The indocyanine green in the nanocomposite photoacoustic imaging agent has a concentration of 5.8-6 μg/mL and preferably 5.8 μg/mL. The pure nanocomposite photoacoustic imaging agent may be stored in vacuum freezing or dispersed in highly pure water at room temperature. Measured by a laser particle size analyzer through the dynamic scattering principle, the nanocomposite photoacoustic imaging agent entrapped by indocyanine green has a particle diameter of 272.0 nm, and a surface electric potential of −5.98 mV. The clear images of the microscopic surface and the fine structure of the nanocomposite photoacoustic imaging agent can be obtained and analyzed by a scanning electron microscope or a transmission electron microscope. Compared with a control of a non-entrapped nanocomposite photoacoustic imaging agent, the imaging and developing effects of the nanocomposite photoacoustic imaging agent of the present invention shows much better contrast and resolution of the photoacoustic imaging in a photoacoustic field. A stronger photoacoustic response signal is generated in a very low concentration, and thus the invention may be applied in the fields of biomarker and medical imaging.

I claim:

1. A polybutylacrylate-based nanocomposite photoacoustic imaging agent, comprising polybutylacrylate as a main component, and methylene blue or indocyanine green as an entrapped component.

2. The polybutylacrylate-based nanocomposite photoacoustic imaging agent as claimed in claim 1, wherein the entrapped component is methylene blue with a particle diameter of 525.1 nm and an electric potential of −4.52 mV.

3. The polybutylacrylate-based nanocomposite photoacoustic imaging agent as claimed in claim 2, wherein the methylene blue in the nanocomposite photoacoustic imaging agent has a concentration of 7.6 μg/mL.

4. The polybutylacrylate-based nanocomposite photoacoustic imaging agent as claimed in claim 1, wherein the entrapped component is indocyanine green with a particle diameter of 272.0 nm and an electric potential of −5.98 mV.

5. The polybutylacrylate-based nanocomposite photoacoustic imaging agent as claimed in claim 4, wherein the indocyanine green in the nanocomposite photoacoustic imaging agent has a concentration of 5.8-6 μg/mL.

6. A manufacturing method of the polybutylacrylate-based nanocomposite photoacoustic imaging agent as claimed in claim 1, comprising the steps of: a) putting 1.5 mL of butyl acrylate, 100 mL of dextran solution, 6.4 mg of methylene blue or 4.9 mg of indocyanine green or in their equivalent proportion into a reaction system;
b) adjusting the pH value of the reaction system pH to 2.0˜3.0, and then adding dilute sodium hydroxide solution to terminating the reaction after mixing at room temperature;
c) setting the reaction system still for 4˜7 hours before transferring the reaction solution to a centrifuge tube for a centrifugal purification;
d) discarding a supernatant liquid, rising with highly pure water, vortexing for several times, and repeating the centrifuge steps for at least two times; and
e) obtaining the nanocomposite photoacoustic imaging agent after a centrifugal concentration.
